(19) United States
(12) Patent Application Publication
(10) Pub. No.: US 2008/0161807 A1
(43) Pub. Date: Jul. 3, 2008

(54) Method and apparatus for targeting blind holes in intramedullary rods

(76) Inventor: Gordon L. Levin, Los Gatos, CA (US)

Correspondence Address: Crockett & Crockett;Suite 400, 24012 Calle De La Plata, Laguna Hills, CA, 92653, US

(21) Appl. No.: 11/648,083

(22) Filed: Dec. 29, 2006

(51) Int. Cl. A61B 17/90

(52) U.S. Cl. 606/64

(57) ABSTRACT

A device and method for locating and drilling through a bone to engage the transverse blind holes in an intramedullary nail. The method is accomplished with a radio-opaque target secured to a patient using a strap or any other suitable binding.

### FIELD OF THE INVENTIONS

The inventions described below relate to the field of medicine and more specifically to targeting, drilling, and engaging blind holes in intramedullary nails.

### BACKGROUND OF THE INVENTIONS

Conventional repair of broken long bones is accomplished by placement of an intramedullary rod or nail in the intramedullary canal of the bone. Securing the nail into the bone is generally accomplished with transverse screws. Locating the transverse holes in the proximal end of the nail may be accomplished with a jig or guide secured to the proximal end of the nail or hand held guides. Locating the transverse holes in the distal end of the nail has been the subject of many proposed solutions secured to the proximal end of the intramedullary nail. The conventional guide solutions suffer from flexure of the guide owing to the distance from the point of attachment to the nail, or from bending or twisting of the intramedullary nail during insertion. These problems lead to surgeons having to “eyeball” the blind holes in the distal end of the nail which requires the surgeon to be in close proximity to the radiation while using the fluoroscopy. This inexact procedure increases operating time, risk of multiple holes drilled into the bone weakening the structure, and increased radiation as well as frustration to the surgeons.

### SUMMARY

The methods and devices described below facilitate placement of intramedullary nails. A method for locating and drilling through a bone to engage the transverse blind holes in an intramedullary nail is accomplished with a radio-opaque target secured to a patient using a strap or any other suitable binding. The radio-opaque target is surrounding a drill channel or port. Iterative fluoroscope images accompanied by adjustment of the target in relation to the imbedded intramedullary nail orients the drill channel through the target with a first hole in the intramedullary nail. An incision permits the surgeon to observe the tip of a drill bit or burr inserted through the target in relation to the bone to be drilled. The first hole is drilled through the bone engaging the first transverse hole in the intramedullary nail. The radio-opaque target may be removed while leaving the drill bit in place. A drill guide may placed with a first hole engaging the first drill bit and a second hole to accept a second drill bit. The drill guide is secured to the patient using a strap, and the second drill bit is used to drill a second hole through the bone engaging the second transverse hole in the intramedullary nail.

### BRIEF DESCRIPTION OF THE DRAWINGS

FIG. 1A is a perspective view of a target block.

FIG. 1B is a top view of the target block of FIG. 1A.

FIG. 1C is a side view of the target block of FIG. 1A.

FIG. 2A is a perspective view of a drill guide.

FIG. 2B is a top view of the drill guide of FIG. 2A.

FIG. 2C is a side view of the drill guide of FIG. 2A.

FIG. 3 is a perspective view of a patient with a target block secured.

FIG. 4 is a perspective view of a patient with a drill bit inserted through a secured target block.

FIG. 5 is a perspective view of a patient with first and second drill bits inserted through a secured drill guide

FIG. 6A is a perspective view of a target and drill guide.

FIG. 6B is a top view of the target and drill guide of FIG. 6A.

FIG. 6C is a side view of the target and drill guide of FIG. 6A.

FIG. 7 is a perspective view of a patient with a target and drill guide secured.

### DETAILED DESCRIPTION OF THE INVENTIONS

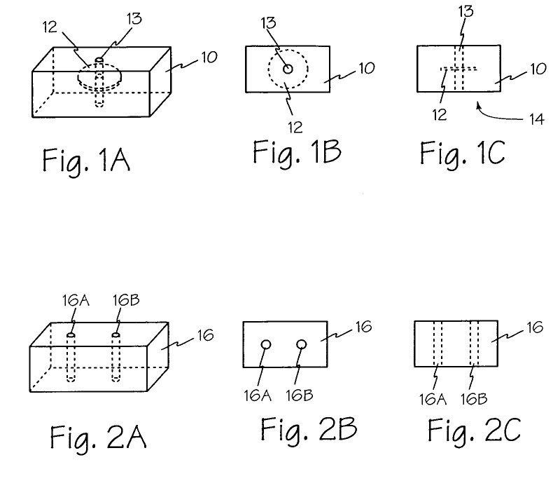

In FIGS. 1A, 1B and 1C target block 10 is illustrated with radio-opaque target 12 surrounding drill port 13. Target block 10 may be secured to a patient with bottom surface 14 against the patient's skin or tissue. Target block 10 may be secured to a patient using a strap or any other suitable technique permitting adjustment of the position to achieve proper orientation of drill port 13 with a transverse hole or holes such as first hole 52 in intramedullary nail 55 of FIG. 7.

Target block 10 may be made of any material suitably transparent to fluoroscopy and radio-opaque target 12 may have any suitable shape affording accurate alignment of drill port 13.

Referring now to FIGS. 2A, 2B and 2C, drill guide 16 includes two or more drill ports such a drill ports 16A and 16B. The relative positions and orientations of drill ports 16A and 16B should be arranged to enable drill bits inserted through the drill ports to engage the transverse holes of the intramedullary nail or other device. Thus it may be necessary to have drill guides such as drill guide 16 specifically for each type of intramedullary nail or other device to be used.

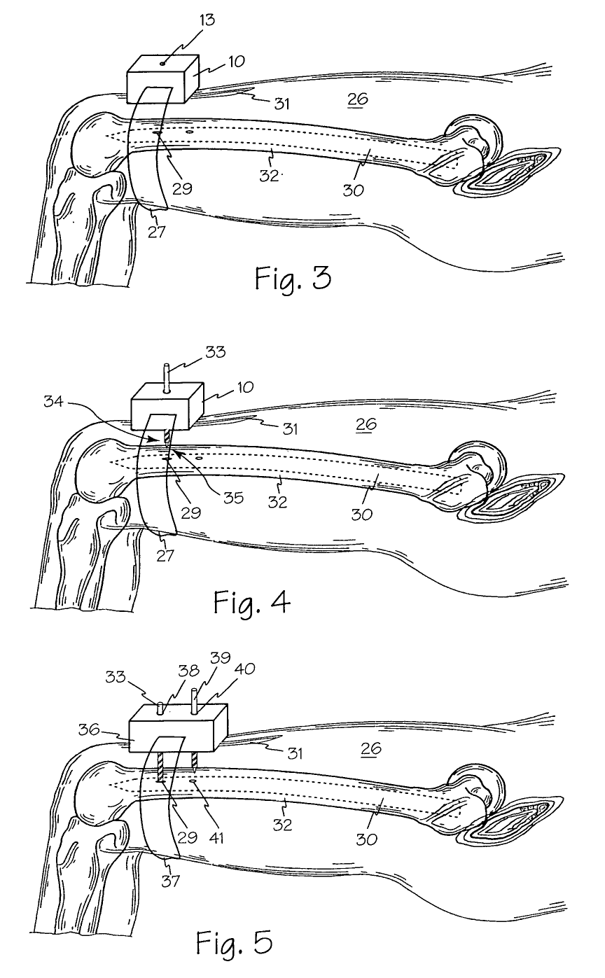

In use, target block 10 may be adjustably secured to patient 26 using strap 27 as shown in FIG. 3. Using fluoroscopy, target block 10 is iteratively adjusted to align drill port 13 with first transverse hole 29 of intramedullary nail 30. Incision 31 is made to permit direct observation of bone 32.

Referring now to FIG. 4, burr or first drill bit 33 may be inserted through drill port 28 until drill tip 34 contacts bone 32. The surgeon may observe location or contact point 35 at which drill tip 34 contacts bone 32 to ensure proper alignment. Thus when a drill motor engages first drill bit 33 any misalignment of the drill with the contact point may be corrected. A drill motor or other source of motive force may then turn first drill bit 33 until the drill bit traversed bone 32 and intramedullary nail 30 through first transverse hole 29. First drill bit 33 is left engaging bone 32 and intramedullary nail 30, and target block 10 is removed.

Referring now to FIG. 5, drill guide 36 is secured to patient 26 using strap 37 with first drill port 38 engaging first drill bit 33, and strap 37 is tightened to secure the drill guide to the patient. Second drill bit 39 is inserted through second drill port 40 and is generally aligned to bone 32 by observation through incision 31. A drill motor or other source of motive force may then turn first drill bit 39 until the drill bit traversed bone 32 and intramedullary nail 30 through second transverse hole 41.

The target block and drill guide and method discussed above, and or the combination target guide and method are illustrated with regard to a human femur. The present disclosure may also be applied to repair of any suitable long bone such as tibia, fibula, humerus etc. and may also be applied to animals.

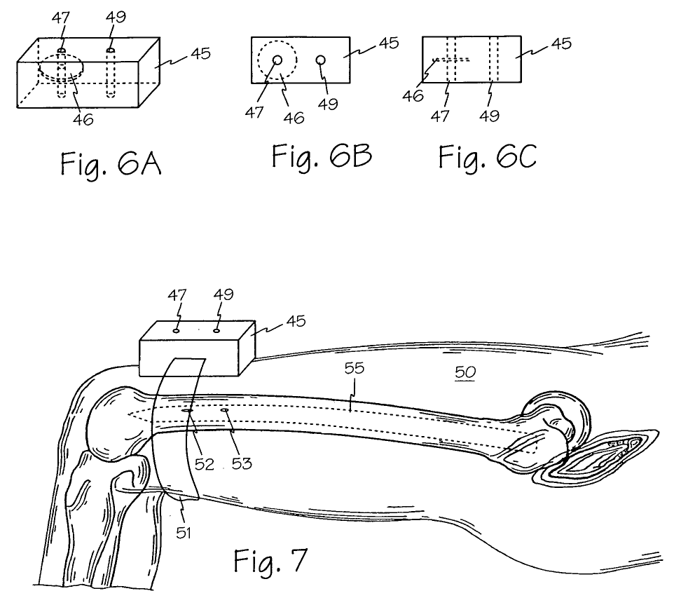

Alternatively, a combination target and drill guide may be used. Combination target guide 45 as shown in FIGS. 6A, 6B and 6C includes a radio-opaque target 46 surrounding a first drill port 47. One or more secondary drill ports such as second drill port 49 may also be available.

As illustrated in FIG. 7, combination target guide 45 may be secured to a patient such as patient 50 using any suitable and adjustable technique such as strap 51. Combination target guide 45 is oriented to align first drill port 47 and second drill port 49 with first hole 52 and second hole 53 respectively, of intramedullary nail 55.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A method of securing blind holes in an intramedullary nail comprising the steps:
aligning two or more pieces of bone forming an intramedullary canal;
inserting an intramedullary nail into the intramedullary canal of the aligned two or more pieces of bone;
movably securing a radio-opaque target on one or more layers of tissue surrounding the aligned two or more pieces of bone;
aligning the radio-opaque target with a first blind hole in the intramedullary nail using fluoroscopy;
securing the radio-opaque target in alignment using a securing strap;
inserting a first drill bit through the aligned radio-opaque target and drilling a hole through at least one of the aligned two or more pieces of bone and the first blind hole of the intramedullary nail;
removing the radio-opaque target;
securing in place over the first drill bit a drill guide having at least two holes, one of the holes engaging the first drill bit; and
inserting a second drill bit through the second hole of the secured drill guide and drilling a hole through at least one of the aligned two or more pieces of bone and a second blind hole of the intramedullary nail.

2. A method of securing blind holes in an intramedullary nail comprising the steps:
inserting an intramedullary nail into the intramedullary canal of a bone;
movably securing a radio-opaque target on one or more layers of tissue surrounding the bone;
aligning the radio-opaque target with a first blind hole in the intramedullary nail using fluoroscopy;
securing the radio-opaque target in alignment using a securing strap;
inserting a first drill bit through the aligned radio-opaque target and drilling a hole through the bone and the first blind hole of the intramedullary nail;
removing the radio-opaque target;
securing in place over the first drill bit a drill guide having at least two holes, one of the holes engaging the first drill bit; and
inserting a second drill bit through the second hole of the secured drill guide and drilling a hole through the bone and a second blind hole of the intramedullary nail.
